(19) United States
(12) Patent Application Publication
(10) Pub. No.: US 2025/0187437 A1
(43) Pub. Date: Jun. 12, 2025

(54) ENHANCED REALITY SYSTEM FOR A VEHICLE AND METHOD OF USING THE SAME

(71) Applicant: FCS US LLC, Auburn Hills, MI (US)

(72) Inventor: John W Haley, Rochester, MI (US)

(21) Appl. No.: 18/536,510

(22) Filed: Dec. 12, 2023

(51) Int. Cl. B60K 35/28; A61B 3/028; B60K 35/233; B60K 35/235

(52) U.S. Cl. CPC B60K 35/285; A61B 3/028; B60K 35/233; B60K 35/235; B60K 2360/176; B60K 2360/177

(57) ABSTRACT

An enhanced reality system for a vehicle having an enhanced reality headset with a headset display, control unit, and power source. The enhanced reality headset displays a video of an upcoming road segment with one or more pieces of supplemental display information. One potential drawback of such a system involves blind spots within a display area, referred to as “visual impairment zones,” which are unique to each driver. The enhanced reality system and method disclosed herein are configured to administer a vision test for each driver, identify any visual impairment zones unique to that driver, determine if any pieces of supplemental display information fall within a visual impairment zone and, if so, move the supplemental display information out of the visual impairment zone so that it is more visible to the driver. An enhanced reality profile may be maintained for each driver.

### FIELD

The present disclosure relates to an enhanced reality system for a vehicle and, more particularly, to an enhanced reality headset where supplemental display information that is overlaid on top of video of an upcoming road segment has been moved or shifted to account for visual impairment zones that are particular to each user.

### BACKGROUND

Enhanced reality technologies, such as virtual reality and augmented reality, are increasingly becoming popular and are now used in a variety of applications. It may even be possible to use such technologies when driving a vehicle.

One potential drawback of such technologies involves blind spots within display areas of enhanced reality headsets, which are unique to each driver. Not all drivers have such blind spots, but those who do may find it difficult to see and interpret certain pieces of information if displayed in those blind spots.

It is, therefore, an object of the present application to provide an enhanced reality system and method that sufficiently addresses and overcomes the preceding drawback.

### SUMMARY

In at least some implementations, there is provided a method of operating an enhanced reality system for a vehicle, comprising the steps of: providing an enhanced reality headset to be worn by a driver of the vehicle, the enhanced reality headset is configured to display a video of an upcoming road segment and supplemental display information overlaid on top of the video; administering a vision test to the driver, the vision test is administered while the driver is wearing the enhanced reality headset and identifies at least one visual impairment zone that is specific to the driver; determining if the supplemental display information overlaid on top of the video is located within the visual impairment zone; when the supplemental display information overlaid on top of the video is located within the visual impairment zone, moving the supplemental display information to a new location where it is easier for the driver to see; and displaying the video of the upcoming road segment and the supplemental display information overlaid on top of the video on the enhanced reality headset, wherein the supplemental display information is in the new location.

In at least some implementations, there is also provided an enhanced reality system for a vehicle, comprising: an enhanced reality headset that includes a headset display for displaying a video of an upcoming road segment and supplemental display information overlaid on top of the video, a headset control unit electronically coupled to the headset display for providing headset input, and a headset power source electronically coupled to the headset display and the headset control unit for providing power, wherein the enhanced reality system is configured to: administer a vision test to a driver of the vehicle, the vision test is administered while the driver is wearing the enhanced reality headset and identifies at least one visual impairment zone that is specific to the driver; determine if the supplemental display information overlaid on top of the video is located within the visual impairment zone; move the supplemental display information to a new location when the supplemental display information overlaid on top of the video is located within the visual impairment zone; and display the video of the upcoming road segment and the supplemental display information overlaid on top of the video on the headset display, where the supplemental display information is in the new location.

Further areas of applicability of the present disclosure will become apparent from the detailed description, claims and drawings provided hereinafter. It should be understood that the summary and detailed description, including the disclosed embodiments and drawings, are merely exemplary in nature intended for purposes of illustration only and are not intended to limit the scope of the invention, its application or use. Thus, variations that do not depart from the gist of the disclosure are intended to be within the scope of the invention.

### BRIEF DESCRIPTION OF THE DRAWINGS

FIG. 1 is a schematic block diagram of an enhanced reality system for a vehicle;

FIG. 2 is a flowchart of an enhanced reality method that may be used with the system from FIG. 1;

FIG. 3 is a schematic illustration of an enhanced reality headset that may be used with the method of FIG. 2, where the headset is being used to administer a vision test; and

FIG. 4 is a schematic illustration of an enhanced reality headset that may be used with the method of FIG. 2, where the headset is being used to display an upcoming road segment with supplemental display information overlaid on the video.

### DETAILED DESCRIPTION

Referring in more detail to the drawings, there is described an enhanced reality system for a vehicle, as well as a method of operating the same. The enhanced reality system includes an enhanced reality headset that, when worn by a driver, displays a video of an upcoming road segment with one or more pieces of supplemental display information, such as traffic warnings or speed limits, overlaid on top of the video. The combined display, including the video and the overlaid pieces of supplemental display information, provides the driver with a more immersive and complete visual experience so that they can safely and confidently drive the vehicle in an enhanced reality environment. One potential drawback of enhanced reality systems involves blind spots within a display area, referred to here as “visual impairment zones,” which are unique to each driver. In order to address such drawbacks, the enhanced reality system of the present application administers a vision test for each driver, identifies any visual impairment zones unique to that driver, determines if any pieces of supplemental display information fall within a visual impairment zone and, if so, moves or shifts the supplemental display information out of the visual impairment zone so that it is more visible to the driver. The system may then save an enhanced reality profile for each driver that includes information regarding the visual impairment zones, the supplemental display information that has been moved, etc. so that the next time the driver uses the system, the enhanced reality headset will provide a combined display that is customized for that driver.

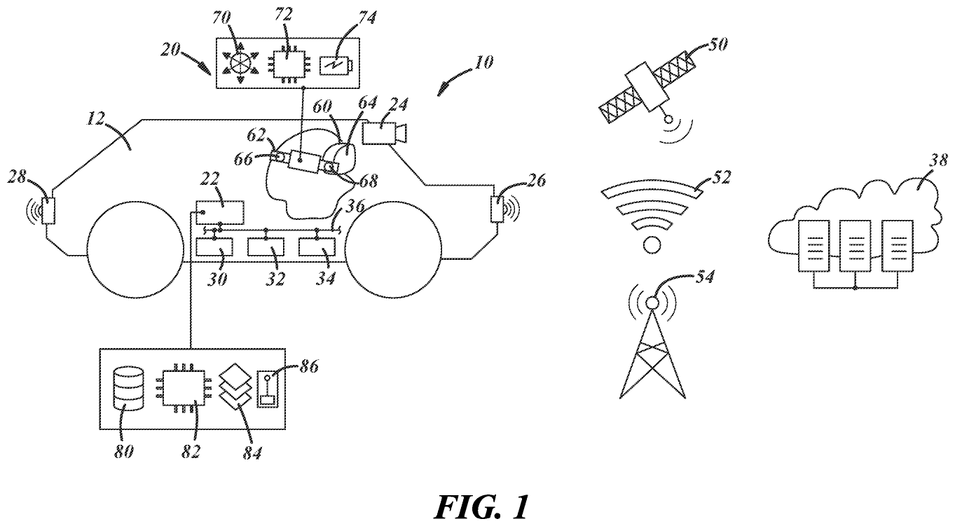

Turning now to FIG. 1, there is shown an example of an enhanced reality system 10 that is incorporated within a vehicle 12 and includes an enhanced reality headset 20 and an enhanced reality module 22. Enhanced reality system 10 may further include and/or interface with various vehicle-mounted sensors 24-28, various vehicle-mounted modules 30-34, a vehicle communication network 36, and a cloud-based backend system 38 that can communicate with the vehicle via some combination of a satellite-based communication network 50, a WiFi-based communication network 52, a cellular-based communication network 54 and/or some other type of communication network. It should be appreciated that any suitable type of wireless communication network, system and/or technology may be employed to connect vehicle 12 to the cloud-based backend system 38, to other vehicles and/or to roadside sensors and devices, and that the present system and method are not limited to any particular type.

Enhanced reality headset 20 provides a driver or other user with an immersive enhanced reality environment where a combined display including both video of an upcoming road segment, as well as various pieces of supplemental display information, are presented in the driver's field of view. According to one example, the enhanced reality headset 20 includes a headset frame 60, a headset fastener 62, a headset display 64, a headset speaker 66 (optional), a headset microphone 68 (optional), one or more headset sensor(s) 70, a headset control unit 72, and a headset power source 74. It should be appreciated that the enhanced reality headset 20 of the present application is not limited to any particular mechanical, electrical and/or software configuration, as any suitable configuration or arrangement may be used and the following description is just one possibility.

Headset frame 60 acts as a frame or housing for the headset and may be mechanically connected to the headset fastener 62 and the headset display 64, as well as any suitable combination of components 66-74, depending on the configuration. For example, headset speaker(s) 66, microphone 68, sensor(s) 70, control unit 72 and/or power source 74 may be directly incorporated or built into the headset frame 60 such that they are integrated features, or they may be separate stand-alone components. According to the schematic illustration in FIG. 1, the headset frame 60 is shaped as a somewhat standard frame, such as those typically found in ski goggles or the like, and is designed to surround the headset display 64 in order to retain and hold it in place. It is also possible for the headset frame 60 to have a different shape, including one more along the lines of a typical virtual reality (VR) headset or even a pair of eye glasses.

Headset fastener 62 secures the headset on the driver's head and may include adjustable straps, buckles, latches, fasteners, etc. that go around and/or over top of the driver's head. The headset fastener 62 can mechanically connect to the headset frame 60, as well as any suitable combination of components 64-74, depending on the configuration. To explain, it is possible for one or more headset speaker(s) 66, microphone 68, sensor(s) 70, control unit 72 and/or power source 74 to be directly incorporated into straps or other components of the headset fastener 62, or for these devices to be attached to the outside of such straps. In the example of FIG. 1, a headset speaker 66 and microphone 68 are built into a strap of the headset fastener 62 (e.g., speaker 66 is at a rearward location near the driver's ear, and microphone 68 is at a forward location closer to the driver's mouth), whereas a headset sensor 70, control unit 72, and power source 74 are part of a separate unit that is attached to a side of the strap.

Headset display 64 is an electronic display inside the headset that can present the driver with video of an upcoming road segment, as well as supplemental display information overlaid on top of the video. The video may be provided from one or more forward facing cameras 24 and/or other sensors mounted on the vehicle. Presenting this combined display in real-time enables the driver to experience an enhanced reality environment where they can confidently drive the vehicle while wearing the enhanced reality headset 20. The headset display 64 may be electronically coupled to the headset control unit 72, the headset power source 74 and/or the enhanced reality module 22 (“electronically coupled,” as used herein, broadly includes wired and wireless connections, direct and indirect connections, data and power connections, etc.). In a preferred example, headset display 64 is electronically coupled to headset control unit 72 to receive headset input and to headset power source 74 to receive power, and it includes one or more high-resolution displays with suitable refresh rates (e.g., the headset display 64 may include a separate display for each eye that superimposes images from one or more video cameras so that a combined image or video is presented to the driver). The headset display 64 may utilize any suitable display technology known in the art, including, but not limited to: fully immersive displays, optical see through displays, video see through displays, liquid crystal displays (LCD), organic light-emitting diode (OLED) displays, high-dynamic-range (HDR) displays, light emitting diode (LED) displays, etc. The headset display 64 of the present application is not limited to any particular type of display or display technology.

Headset sensor(s) 70 may be mounted on the enhanced reality headset 20 and can gather various types of headset data, such as the orientation or pose of the driver's head. The headset sensor(s) 70 may be electronically coupled to the headset display 64, the headset control unit 72, the headset power source 74 and/or the enhanced reality module 22 and can include any suitable combination of accelerometers, gyroscopes, motion sensors, cameras, etc. In one example, headset sensor(s) 70 include one or more sensors that track the orientation or pose of the driver's head, referred to here as “headset data,” so that the system can synchronize video of the upcoming road segment to the direction in which the driver is looking when the video is shown on the headset display 64. This way, when the driver turns their head to the left, the video shown on the headset display 64 correspondingly turns to the left, and when they turn their head to the right, the video turns to the right. The video displayed on the headset display 64 may be provided by the vehicle mounted camera 24, by camera(s) mounted on the headset 20 or by some other source. The headset sensor(s) 70 may also include one or more sensors to detect the direction, position and/or state of the driver's eyes, as this headset data may be needed to administer the vision test and/or provide an enhanced reality environment, as will be explained. Other sensors and sensing elements may be included as well.

Headset control unit 72 may be mounted on the enhanced reality headset and can use data from a variety of sources, both on the headset and around the vehicle, to control certain aspects of the headset display 64. The headset control unit 72 may be electronically coupled to the headset sensor(s) 70 to receive headset data, as well as the enhanced reality module 22, vehicle-mounted sensors 24-28, vehicle-mounted modules 30-34 and/or the cloud-based backend system 38. The headset control unit 72 may include any suitable electronic controller, processor, microprocessor, microcontroller, application specific integrated circuit (ASIC) and/or other electronic processing device, as well as an electronic memory device and a wireless communication device (e.g., a wireless transceiver of some type). Electronic instructions used to implement the method described herein may be saved on a computer-readable storage medium that is part of the electronic memory device in headset control unit 72, and the wireless communication device could be used to communicate with the enhanced reality module 22. The present method and system may assign any appropriate division of processing and/or other tasks between the headset control unit 72 and the enhanced reality module 22. Furthermore, the headset control unit 72 is not limited to any particular configuration and may be a standalone unit that is attached on a side of the headset frame 60 or fastener 62, it may be combined with other electronic components like the headset senso(S) 70 and power source 74 (as shown in FIG. 1), or it may be integrated within the headset frame 60, fastener 62, display 64 and/or the enhanced reality module 22, to cite a few possibilities.

Headset power source 74 provides power or energy to the enhanced reality headset and may include one or more rechargeable battery (ies), non-rechargeable batter (ies) or some other form of an energy storage device. The headset power source 74 may be electronically coupled to the headset display 64, speaker 66, microphone 68, sensor(s) 70 and/or control unit 72. Preferably, the headset power source 74 is small enough to be integrated, either as a combination of devices or by itself, into the headset frame 60, fastener 62 and/or display 64 and have sufficient charge to power the enhanced reality headset for many hours. Wired or wireless charging features could be used to charge the headset power source 74.

Enhanced reality module 22 is installed on vehicle 12 and may be responsible for, among other things, gathering data from around the vehicle and sending data to and/or receiving data from headset control unit 72. The enhanced reality module 22 may include any suitable combination of software and/or hardware resources typically found in such modules, including data storage unit 80, electronic control unit 82, various application(s) 84 and communications unit 86. Enhanced reality module 22 may be a dedicated and standalone module, or it may be part of an instrument panel cluster control module (IPCCM), a body control module (BCM), a telematics control module (TCM), a navigation control module, an infotainment control module, or any other suitable module or device known in the art. It is not necessary for units 80-86 to be packaged in a single integrated electronic module, as illustrated in FIG. 1. Rather, they could be distributed among multiple vehicle electronic modules, they could be stand-alone units, they could be combined or integrated with other units or devices, or they could be provided according to some other configuration. In one possible example, the headset control unit 72 is wholly or partially combined with the enhanced reality module 22 such that the module 22 controls operation of the headset display 64, as opposed to the control unit 72. The enhanced reality module 22 is not limited to any particular architecture, infrastructure or combination of elements, as any suitable module or device may be employed.

Vehicle-mounted sensors 24-28 may include any suitable combination of cameras, radar sensors, laser sensors, lidar sensors, etc. and can provide the enhanced reality system with various types of road data and/or other data. In one example, vehicle-mounted sensor 24 is a forward facing camera that is mounted on the vehicle 12, captures video of the upcoming road segment, and is electronically coupled to the headset control unit 72, either directly or indirectly through the enhanced reality module 22 and/or some other device. This data provided by sensors 24-28 may be used to further enrich the enhanced reality environment provided by the present system and method.

Vehicle-mounted modules 30-34 may include any suitable combination of electronic modules typically found on vehicles, including the several modules listed above. As with their sensor counterparts 24-28, vehicle-mounted modules 30-34 may provide the enhanced reality system with various types of vehicle data and/or other data to help facilitate and improve the enhanced reality environment created by the present system and method.

Vehicle-mounted communication network 36 may connect various sensors, units, devices, modules and/or systems in vehicle 12 and can include a network and/or bus, such as a controller area network (CAN) or a local interconnect network (LIN). Communication network 36 may be wholly or partially wired or wireless. In one example, communication network 36 connects enhanced reality module 22 with the vehicle-mounted sensors 24-28, the vehicle-mounted modules 30-34, and possibly the enhanced reality headset 20 (e.g., with a wireless connection).

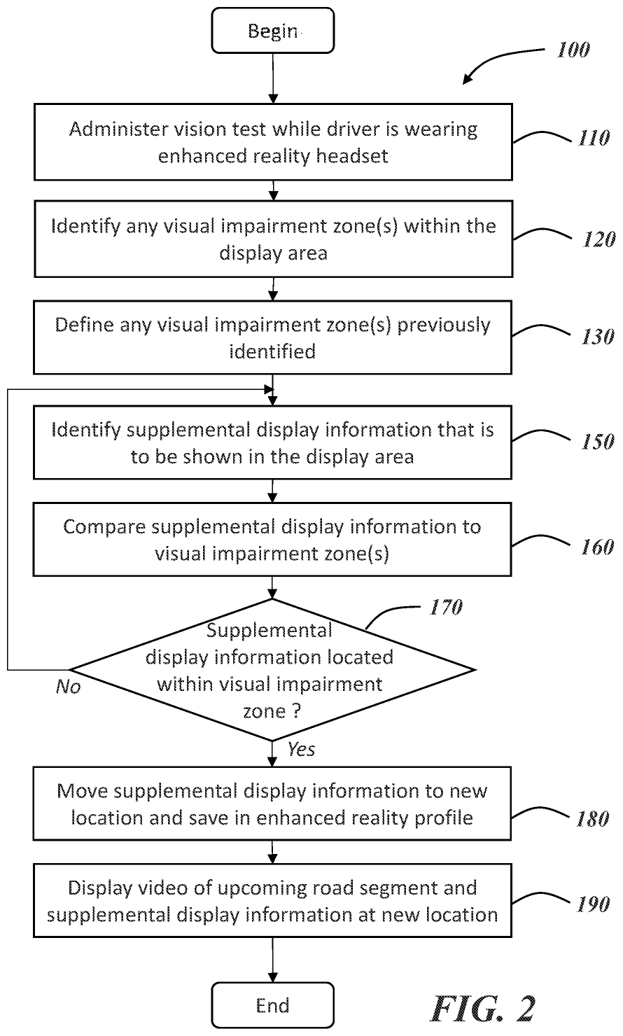

Turning now to FIG. 2, there is shown an example of a method 100 that may be used to operate the enhanced reality system 10. In general, method 100 administers a vision test for a driver, identifies any visual impairment zones that are specific or unique to that driver, adjusts the location of any supplemental display information located within a visual impairment zone, and saves this information in an enhanced reality profile for that particular driver. The term “enhanced reality,” as used herein, broadly includes any type of reality technology (e.g., augmented reality, virtual reality, etc.) that enhances and/or replaces a real-life environment with a simulated one. In a preferred example, the enhanced reality system and method of the present application utilize “augmented reality,” which is a type of enhanced reality technology that augments or supplements the visual experience of the driver by overlaying various types of supplemental display information, such as traffic warnings or speed limits, on top of live video of the upcoming road segment. The combined display, along with optional audio information, provides the driver with an enhanced reality environment, in which they can safely and confidently drive the vehicle. It should be noted that the terms “driver” and “user” are used interchangeably, as the enhanced reality system and method of the present application is designed for a driver but may be utilized by passengers and other users as well.

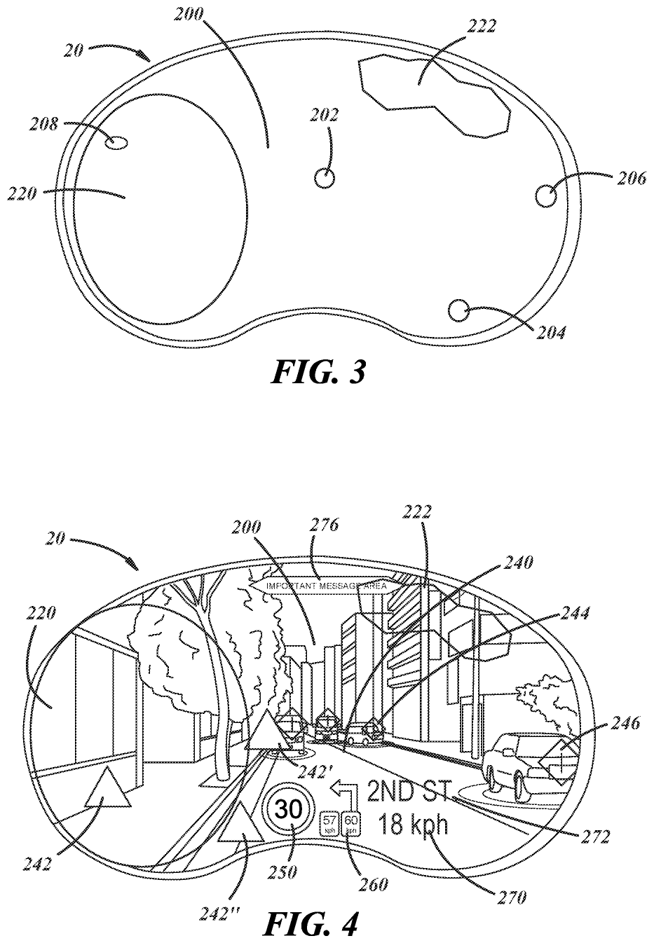

Starting with step 110, the method administers a vision test to each driver or user of the system. The vision test is conducted while the driver is wearing the enhanced reality headset 20 and is designed to identify any visual impairment zones that may be affecting the vision of that particular driver, as will be explained in conjunction with FIG. 3. There are a number of different vision tests that could be administered in step 110, including the Humphrey vision test, which evaluates a driver's vision within a display area 200 that is part of the headset display 64. This test is performed on one eye at a time (the other eye is covered). During this test, the driver focuses on a central light 202 while side lights 204-208 appear in different parts of the display area 200, oftentimes towards the periphery. The side lights 204-208 may be blinking, they may have different light intensities, they may have difficult colors, they may be different sizes, they may move within the display area 200, etc. The driver is then asked to press a button and/or otherwise indicate when they see a side light 204-208 enter their peripheral field-of-view. In the case of the system 10, the driver may provide their feedback by engaging a button on the enhanced reality headset 20, by engaging a button on the steering wheel of vehicle 12, by providing a verbal indication which is captured by headset microphone 68 or some other human-machine-interface (HMI), or by indicating according to some other suitable means. It is desirable for the driver or other user to continue focusing their eye on the central light 202 during the test, as moving their eye can decrease the accuracy or reliability of the test. To help ensure the accuracy of this test, it is possible for one or more of the headset sensor(s) 70 to include a small camera that is directed at the driver's eye and captures its position and/or direction in which it is looking.

Another example of a potential vision test is the Goldmann vision test, which involves moving progressively dimmer lights from a periphery of the display area 200 towards a center of the display area in order to identify and map the location where the light is first seen by the driver. It should be appreciated that the Humphrey and Goldmann vision tests are just two potential vision tests that may be administered, but that step 110 is not so limited. Other tests and/or features may be employed by step 110 in order to improve its accuracy or reliability.

Next, the method identifies any visual impairment zones within the display area, step 120. According to one example, this step may compare the driver's responses gathered in step 110 to typical or expected responses and then analyze the results to identify any visual impairment zones 220-222. The term “visual impairment zone,” as used herein, broadly includes an area or region within the display area 200 where a particular driver has some form or degree of vision loss. Step 120 may determine that a particular driver has no visual impairment zones, a single visual impairment zone, or multiple visual impairment zones. Moreover, the size, shape, orientation, location and/or severity of visual impairment zones may vary for a particular driver (e.g., a first visual impairment zone 220 may be large, circular or oval and located at an outer peripheral region within display area 200, while a second zone 222 may be small, irregular in shape and located towards an upper peripheral region). The exact nature of each visual impairment zone 220-222 is unique to each driver, thus, explaining why each driver is individually tested and why a unique enhanced reality profile is maintained. Skilled artisans will appreciate that the size, shape and/or other characteristics of a visual impairment zone may differ from the non-limiting examples shown in the drawings, as they are only provided for purposes of illustration.

In step 130, the method defines any visual impairment zone(s) that was identified in the previous step and does so according to one of a number of different techniques. Defining a visual impairment zone typically involves establishing the size, shape and/or location of the zone so that the method can later determine if any supplemental display information falls within that zone, as will be explained. In one example, the display area 200 includes a two-dimensional array or matrix of pixels that are arranged in columns and rows in a grid-like fashion, and step 130 defines each visual impairment zone 220-222 in terms of the column and row information of the pixels that make up that zone. In this example, each of the visual impairment zones 220 and 222 may include hundreds, thousands, tens of thousands, hundreds of thousands or even millions of pixels that make up a so-called restricted pixel matrix. The pixel information may be stored in some type of suitable data storage unit in the enhanced reality headset 20 and/or module 22. Instead of identifying all of the pixels that make up each visual impairment zone, step 130 may simply identify pixels that make up the boundary or border of each zone, thereby reducing the amount of information that needs to be stored but still adequately defining each zone. In a different example, step 130 identifies a center or middle of each visual impairment zone and then establish a radius and/or other dimension(s) that defines the zone in relative terms based on the center point. Other suitable techniques may be used to define the visual impairment zones and step 130 is intended to cover such techniques.

At this point, the method has visually tested the driver and identified and defined any visual impairment zone(s). Steps 110-130 are performed when the driver is not driving the vehicle (e.g., they could be performed when the driver is initially establishing their settings and/or preferences for the enhanced reality system 10). The next several steps determine if any supplemental display information, such as traffic warnings or speed limits that are part of the enhanced reality environment, falls within a visual impairment zone. If so, the method can move or shift such supplemental display information out of the visual impairment zone (e.g., with the use of software techniques), thereby providing the driver with an enhanced reality environment that is somewhat customized to their vision. Steps 150-190 could be performed contemporaneously with steps 110-130 as part of the same setup process, or they could be performed at a later time when the driver is in the process of actually using the enhanced reality headset 20. The present method is not limited to the particular combination and/or sequence of steps shown and described herein, as those steps are simply provided for purposes of illustration.

Step 150 identifies supplemental display information that is to be displayed within a display area of the enhanced reality headset while the vehicle is being driven. With reference to FIG. 4, there is shown a non-limiting example of a display area 200 where both live video of an upcoming road segment 240 and several pieces of supplemental display information 242-276 are presented to the driver. The term “supplemental display information,” as used herein, broadly includes any symbols, alphanumeric characters, indicia, words, warnings, messages and/or other information that is not part of the actual live video of the upcoming road segment, but rather is electronically generated information that is overlaid or superimposed on top of the video. Some non-limiting examples of supplemental display information include traffic warnings 242-246, speed limits 250, navigational directions 260, current vehicle information (e.g., vehicle speed 270, location 272, fuel level, engine temperature, tire pressure, etc.), messages 276, infotainment items (e.g., radio, phone calls, etc.), data from the cloud-based backend system 38 (e.g., traffic and road conditions, software updates, system analytics, vehicle maintenance items, etc.), as well as any other electronically generated information intended to improve the driver's enhanced reality environment. The supplemental display information may include a single piece of information or multiple pieces of information, it may include fixed pieces of information that stay in one location or moving pieces of information that change locations, or it may include static pieces of information that do not vary in terms of their content or color or brightness or it could include dynamic pieces of information that do vary, to cite several possibilities. The supplemental display information may be part of a default enhanced reality environment that comes with each headset 20 or it may be customized by each driver based on the type and amount of information they wish to see. In any event, step 150 identifies or determines the supplemental display information that is to be shown in the display area 200.

Next, the method compares the supplemental display information that is to be shown in the display area (i.e., the supplemental display information identified in the previous step) to the previously defined visual impairment zone(s), step 160. The method performs this step in order to determine if any supplemental display information is located wholly or partially within a visual impairment zone where the driver may not be able to adequately see it. According to one example, step 160 gathers the location where each piece of supplemental display information 242-276 is to be shown in the display area 200 (e.g., pixel information in terms of columns and rows) and compares it to the location of the different visual impairment zones 220, 222. The original location of supplemental display information 242 is compared to the location of visual impairment zone 220 and step 170 determines that traffic warning 242 falls wholly within zone 220 located on the left side of the display area 200. Thus, the traffic warning should be moved or shifted out of zone 220, from the original location 242 to a new location 242′ where the driver can see it better, as will be explained. Display information 244-272, on the other hand, does not fall wholly or partially within any visual impairment zones 220, 222 and step 170 determines that this information does not need to be moved or shifted. The method may then loop back to step 150 for continued monitoring.

An interesting situation occurs when supplemental display information, such as message 276, is located partially within a visual impairment zone. In this scenario, step 170 may determine that the message 276 should be completely shifted out of the visual impairment zone 222, it may decide to shift the message partially out of the visual impairment zone, or it may decide to leave the message where it is. A number of different analytical techniques may be used by step 170 to make the aforementioned decision. For instance, the method could determine how much of the supplemental display information is located within the visual impairment zone-if only a small portion of the message is located within the zone, as is the case with message 276 and zone 222, then step 170 decide that there would be little to gain from moving the message and that it should stay in its original location. If a larger portion (e.g., more than 50%) of the supplemental display information falls within the confines or boundaries of the visual impairment zone, then step 170 may decide to move that information to a new location.

In another embodiment, step 170 may consider the severity of the visual impairment zone (i.e., the degree of vision loss in that area) and/or the criticality of the supplemental display information. To explain, if the driver has severe vision loss in visual impairment zone 222, for instance, as detected by the previously administered vision test, then step 170 may decide to move message 276 out of that zone, even though only a small portion of the message is in the impaired area. If the vision loss in zone 222 was only minor, and not severe, then step 170 may decide to leave the message 276 in its original location. Of course, if the visual impairment is so severe, the method could inform the driver that there are not enough unimpaired areas in the display area 200 to provide the supplemental display information. In yet another embodiment, if a particular piece of supplemental display information, like warning 246 which indicates the presence of a parked car, is considered critical, then step 170 may decide to move that warning out of a visual impairment zone, even if the vision loss in that zone is not severe. Other considerations and factors may also be used by the method when determining which supplemental display information to move and which to keep in its original location. If the severity of the visual impairment zone and/or the criticality of the supplemental display information is significant enough, the method may decide to augment visual warnings to the driver with one or more audible, tactile and/or other warnings.

Step 180 moves the supplemental display information from an original location to a new location and then saves the various information described herein in an enhanced reality profile. The location of the supplemental display information, whether it be the original or new location, is typically chosen so as to not distract the driver or obscure their field of view. This is way the majority of pieces of supplemental display information 242-272 are positioned somewhat around the periphery of the display area 200 and not in the middle. Of course, some types of supplemental display information, like those identifying or indicating obstacles in the road, may need to be located in a position that is dictated by the location of the obstacle. In one example, step 180 moves the supplemental display information to a new location that is as close as possible, yet is still out of the way and/or accurately indicates the position of the obstacle for which it is warning. The triangular warning in FIG. 4, which indicates another vehicle in the blind spot of vehicle 10, is moved from its original location 242 to a new location 242′ that is as close as possible (e.g., it is just outside the boundary of visual impairment zone 220), yet is still in close enough proximity to intuitively inform the driver of a vehicle in their driver's side blind spot. In a different embodiment, the triangular warning could be moved to a new location 242″, which is positioned towards the bottom of the display area 200, at an even more out of the way location. In the event that the driver was dissatisfied with the new location of the information, the method may offer the driver the option of declining and/or changing the new location.

The results of the vision test, the location and characteristics of the visual impairment zone(s), the original and new locations of the supplemental display information, as well as any other pertinent information (e.g., information related to the driver's identification, enhanced reality environment preferences or settings, etc.) may be saved by step 180 in an enhanced reality profile for that particular driver. This way, when the driver puts on the enhanced reality headset 20 in the future, system 10 can automatically recognize the driver and present them with an enhanced reality environment that has been customized for them. Saving a different profile for each driver also enables a number of different drivers and/or passengers to use the same enhanced reality headset. The enhanced reality profile may be saved at enhanced reality headset 20 and/or module 22, to cite a few possibilities.

At this point, the method may check to see if there are any other pieces of supplemental display information located within visual impairment zones (not shown in the flowchart), or the driver may start using the enhanced reality headset 20 to drive vehicle 12. In use, the enhanced reality headset 20 presents the driver with a combined display that includes video of the upcoming road segment and supplemental display information overlaid on top of the video, where at least one piece of supplemental display information has been shifted or moved out of a visual impairment zone to a new location that is easier for the driver to see.

It is to be understood that the foregoing is a description of one or more preferred exemplary embodiments of the invention. The invention is not limited to the particular embodiment(s) disclosed herein, but rather is defined solely by the claims below. Furthermore, the statements contained in the foregoing description relate to particular embodiments and are not to be construed as limitations on the scope of the invention or on the definition of terms used in the claims, except where a term or phrase is expressly defined above. Various other embodiments and various changes and modifications to the disclosed embodiment(s) will become apparent to those skilled in the art. All such other embodiments, changes, and modifications are intended to come within the scope of the appended claims.

As used in this specification and claims, the terms “for example,” “e.g.,” “for instance,” “such as,” and “like,” and the verbs “comprising,” “having,” “including,” and their other verb forms, when used in conjunction with a listing of one or more components or other items, are each to be construed as open-ended, meaning that the listing is not to be considered as excluding other, additional components or items. Other terms are to be construed using their broadest reasonable meaning unless they are used in a context that requires a different interpretation.

What is claimed is:

1. A method of operating an enhanced reality system for a vehicle, comprising the steps of:
providing an enhanced reality headset to be worn by a driver of the vehicle, the enhanced reality headset is configured to display a video of an upcoming road segment and supplemental display information overlaid on top of the video;
administering a vision test to the driver, the vision test is administered while the driver is wearing the enhanced reality headset and identifies at least one visual impairment zone that is specific to the driver;
determining if the supplemental display information overlaid on top of the video is located within the visual impairment zone;
when the supplemental display information overlaid on top of the video is located within the visual impairment zone, moving the supplemental display information to a new location where it is easier for the driver to see; and
displaying the video of the upcoming road segment and the supplemental display information overlaid on top of the video on the enhanced reality headset, wherein the supplemental display information is in the new location.

2. The method of claim 1, wherein the administering step further includes administering either a Humphrey vision test or a Goldmann vision test to the driver while the driver is wearing the enhanced reality headset.

3. The method of claim 1, wherein the administering step further includes administering a vision test to the driver while one or more headset sensor(s) in the enhanced reality headset monitor a direction, position and/or state of the driver's eyes to help ensure accuracy of the vision test.

4. The method of claim 1, wherein the administering step further includes defining the visual impairment zone that was identified in terms of its size, shape and/or location.

5. The method of claim 4, wherein a display area of a headset display includes a two-dimensional array or matrix of pixels arranged in columns and rows in a grid-like fashion, and the size, shape and/or location of the visual impairment zone is defined in terms of pixel information.

6. The method of claim 1, wherein the determining step further includes identifying supplemental display information that is to be shown in a display area of a headset display, comparing an original location of the supplemental display information that is to be shown to the visual impairment zone, and determining if the original location of the supplemental display information that is to be shown is located within the visual impairment zone.

7. The method of claim 1, wherein the determining step further includes determining if an original location of the supplemental display information that is to be shown is located wholly or partially within the visual impairment zone; and
when the supplemental display information is located partially within the visual impairment zone, evaluating what portion of the supplemental display information is located within the visual impairment zone and then determining whether to move the supplemental display information from the original location to the new location based on the evaluated portion.

8. The method of claim 1, wherein the determining step further includes determining a severity of the driver's vision loss in the visual impairment zone and using the severity as a factor in determining if the supplemental display information is located within the visual impairment zone.

9. The method of claim 1, wherein the determining step further includes determining a criticality of the supplemental display information and using the criticality as a factor in determining if the supplemental display information is located within the visual impairment zone.

10. The method of claim 1, wherein the moving step further includes moving the supplemental display information to a new location that is close to an original location, yet is still out of the way so as to not obscure the driver's view.

11. The method of claim 1, further comprising the step of:
saving an enhanced reality profile for the driver, wherein the enhanced reality profile includes information regarding the visual impairment zone, the supplemental display information, and the new location.

12. An enhanced reality system for a vehicle, comprising:
an enhanced reality headset that includes a headset display for displaying a video of an upcoming road segment and supplemental display information overlaid on top of the video, a headset control unit electronically coupled to the headset display for providing headset input, and a headset power source electronically coupled to the headset display and the headset control unit for providing power, wherein the enhanced reality system is configured to:
administer a vision test to a driver of the vehicle, the vision test is administered while the driver is wearing the enhanced reality headset and identifies at least one visual impairment zone that is specific to the driver;
determine if the supplemental display information overlaid on top of the video is located within the visual impairment zone;
move the supplemental display information to a new location when the supplemental display information overlaid on top of the video is located within the visual impairment zone; and
display the video of the upcoming road segment and the supplemental display information overlaid on top of the video on the headset display, where the supplemental display information is in the new location.

13. The enhanced reality system of claim 12, wherein the enhanced reality system further includes: a forward facing camera that provides video of the upcoming road segment and is mounted on the vehicle and an enhanced reality module that is mounted on the vehicle, the forward facing camera is electronically coupled to the enhanced reality module and/or the headset control unit, which in turn is/are electronically coupled to the headset display, wherein the enhanced reality system is configured to display the video of the upcoming road segment provided by the forward facing camera on the headset display.

14. The enhanced reality system of claim 13, wherein the enhanced reality system further includes: a camera that provides video of the upcoming road segment and is mounted in the enhanced reality headset, the camera is electronically coupled to the headset control unit, which in turn is electronically coupled to the headset display, wherein the enhanced reality system is configured to display the video of the upcoming road segment provided by the camera on the headset display.

15. The enhanced reality system of claim 13, wherein the enhanced reality headset further includes: a headset frame that retains the headset display, a headset fastener that secures the enhanced reality headset on the head of the driver, and at least one headset sensor that is electronically coupled to the headset control unit and provides headset data regarding a direction, position and/or state of the driver's eyes, wherein the enhanced reality system is configured to administer the vision test to the driver, based at least partially on the headset data from the headset sensor, while the driver is wearing the enhanced reality headset.
